(19) United States
(12) Patent Application Publication
(10) Pub. No.: US 2005/0119750 A1
(43) Pub. Date: Jun. 2, 2005

(54) Intervertebral prosthesis or nucleus replacement prosthesis

(75) Inventor: Armin Studer, Winterthur (CH)

Correspondence Address: RANKIN, HILL, PORTER & CLARK LLP, 4080 ERIE STREET, WILLOUGHBY, OH, 44094-7836, US

(73) Assignee: Marthys Medizinaltechnik AG, Bettlach (CH)

(21) Appl. No.: 10/509,931

(22) PCT Filed: Apr. 4, 2002
(86) PCT No.: PCT/CH02/00189

(51) Int. Cl. A61F 2/44

(52) U.S. Cl. 623/17.16; 623/17.11

(57) ABSTRACT

An intervertebral disk prosthesis or nuclear prosthesis (1) includes a longitudinal, flexibility-yielding object (2) that can be spiral-wound (2), with a first exterior end (3), a second interior end (4) and a longitudinal central axis (5). The cross-section (10) of the object (2), which is orthogonal towards the central axis (5), is reduced in size towards the second interior end (4).

The invention relates to an intervertebal prosthesis or nuclear prosthesis as per to the generic term of patent claim 1.

Such prostheses are inserted into the intervertebral disk space between two adjacent vertebral bodies, after removal of the damaged natural disk or the damaged nucleus of a disk. The intended objective is to recreate conditions resembling the natural conditions as closely as possible, which means in particular restoring the height of the original disk and thereby restoring the original spacing between two adjacent vertebral bodies.

From the state of the art intervertebral disk prostheses are already known, for example from FR-A-2 712 486 BRESLAVE, whereby a bendable but not flexibility-yielding VELKRO tape is spiral-wound to form a circular disk. To enable winding the tape in a spiral shape, this known prosthesis requires a cylindrical centre piece to which the tape is attached and then wound around it by rotating the centre-piece.

A disadvantage of this known intervertebral disk prosthesis is the transition between the relatively large centre piece and the relatively narrow tape as well as the lacking elasticity-yield of the tape. The size of the centre piece at the same time also determines the size of the entry opening, whereby the latter should be kept as small as possible which, however, is impossible with this known prosthesis.

From EP-A-0 773 008 an intervertebral prosthesis according to the generic term under claim 1 is known. A disadvantage of this known prosthesis is again the relatively large cylindrical centre piece to which the elongated, spiral-shaped object is attached with a linking component of a lesser width which acts as a joint. Handling of the cylindrical centre piece with the successive articulate linking component is difficult and requires effort—precisely because it is shaped to be springy and articulate. Furthermore, the cylindrical centre piece is obtrusive.

The above discussion of the state of the art was merely entered into to describe the context of the invention and does not mean that at the time of this application or its priority, the state of the art had actually been publicised or was public knowledge.

The invention intends to resolve these issues. The invention is based on the challenge of producing an intervertebral disk prosthesis or nucleus prosthesis which due to its geometric shape restores the height of the intervertebral disk, absorbs any occurring acting forces across the entire—preferably convex-shaped —surface and reduces the pressure in the facet joints, diverts forces to the anulus while not aversely affecting the natural movement but supporting it.

The reduced size of the interior end of the intervertebral disk prosthesis or nuclear prosthesis enables easier handling of the instruments. As a result the resistance when drawing the implant into the inserting instrument lessens.

The endplate centre is very thin and can therefore be pushed in relatively easily; on account of the thinner cross section in the centre of the implant, it is designed as a relatively flexible zone, so that the occurring pressures are better absorbed.

The invention solves the task at hand with an intervertebral disk prosthesis featuring the characteristics of claim 1.

The reduction of the cross section which is orthogonal to the central axis, preferably occurs continuously and preferably towards the first exterior end of the object. The width of the object, measured vertically to the central axis, should also be reduced—preferably continuously—beginning from its centre towards the exterior end. In addition, the width may also be reduced towards the interior end, preferably continuously. In this manner an increased flexibility of the instruments for handling the implant is achieved.

In its centre the width of the object is typically wider by 50% to 500%, preferably by 100% to 300% compared with its interior and exterior end. Thereby individual flexibility can be controlled and a larger bearing surface towards the cover plates of the vertebral bodies is achieved.

In a specific embodiment of the invention with the object in a spiral-wound state to the central axis, it features an upper spiral level and a lower spiral level, both of which arched in a convex-shape and suitable for application to the cover plates of two adjoint vertebral bodies. This achieves self-centering of the implant within the concave endplates of the vertebral bodies and a height increase of the application surface as well as a reduction of the specific surface pressures. Overall this results in a better transfer of forces to the anulus and the endplate.

Suitably, in its spiral-wound and unloaded state, the object has a gap which on the one hand facilitates the production of the object and on the other hand guarantees optimum flexibility. The gap should have a width of a minimum of 0.4 mm, preferably a minimum of 0.5 mm. On the other hand the gap should not exceed a maximum width of 1.0 mm, with the preferred maximum being 0.8 mm. Within these ranges an optimum memory effect of the object with spiral winding occurs.

In a specific embodiment of the invention, the object in its spiral-wound state—viewed from the spiral level—features an oval or kidney-shaped shape, preferably with a surface between 250 to 750 mm² measured at the spiral level, which results in an optimum adjustment to the anatomical contributing conditions.

In a preferred embodiment of the invention the object contains a hydrogel or is even exclusively made up of hydrogel. Hydrogels are colloids in which the dispersal phase (colloid) has combined with the continuous phase (water) to form a viscous, gel-like compound, e.g. coagulated silicon acid. Compared with other materials this brings the benefit of releasing water when under pressure and absorbing water with load relief, which means it acts analogous to the natural nucleus.

Suitably, the object is manufactured using an injection-molding process whereby its injection point is preferably located in the area of the second end. For the production this is an advantage in terms of the filling process. Preferably the injection point is placed in a recessed position. Therefore, warping caused by pulling out the needle nozzle, does not occur on the surface but inside the recess.

In a specific embodiment of the invention the first end is designed asymmetrically towards the interior of the spiral. This results in the exterior form of the object with spiral winding to be rounded.

The object can be designed to be X-ray-opaque, preferably by using the additive barium sulfate. This allows checking the current position of the implant and monitoring any possible migration. For the same purpose the object may also contain X-ray-opaque components, preferably in the form of filaments, wires or tiny globes.

In a specific embodiment of the invention the last exterior turn of the spiral, amounting to at least 360° C. of the circumference of the spiral-wound object, has a thinner cross section when compared with the remaining spiral turns. As a result the exterior rim of the implant is more flexible in terms of the function of the implant.

Finally, the exterior end of the object can be fitted with aids suitable for gripping the intervertebral disk prosthesis using an inserting instrument, preferably in the form of indentations or protrusions.

The following describes the invention and extensions of the invention in more detail by means of schematic illustrations of an embodiment of the invention.

It shows

FIG. 1—a view shown in perspective of an intervertebral disk prosthesis in terms of the invention;

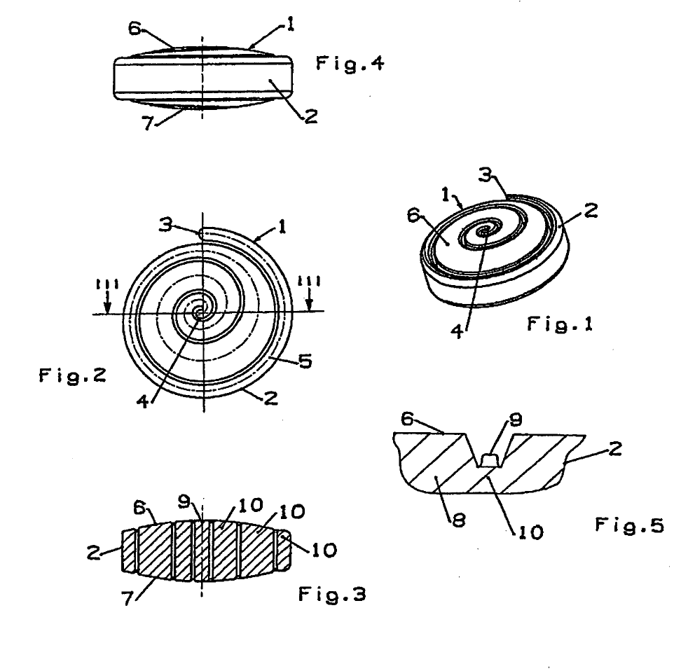

FIG. 2 shows a horizontal cross section through the intervertebral disk prosthesis as per FIG. 1;

FIG. 3 shows a cross section along the line III-III in FIG. 2;

FIG. 4—a side view of the intervertebral disk prosthesis as per FIG. 1;

FIG. 5—an enlarged section of FIG. 3 in the area of the central injection point;

FIG. 6—an enlarged view shown in perspective of the exterior end of the object with spiral winding as per FIG. 1; and

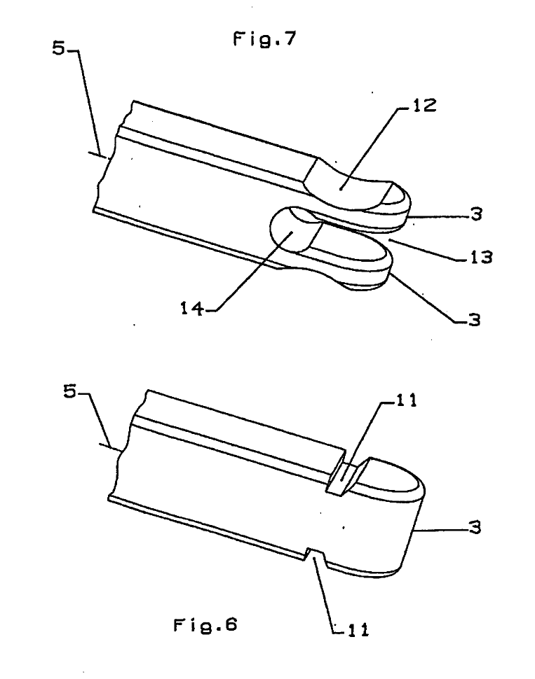

FIG. 7 a variant of the exterior end of the object with spiral winding as per FIG. 1.

The intervertebral disk prosthesis 1 shown in FIG. 1 to 4 consists of a longitudinal, flexibility-yielding object 2 which may be spiral-wound with a first exterior end 3 and a second interior end 4 and a longitudinal central axis 5. The cross section 10 which is orthogonal to the centre axis 5 of the object 2, is reduced continuously—as shown in FIGS. 2 and 3—towards the second interior end 4 as well as towards the first exterior end 3.

The measured width of object 2 in FIG. 2 at its interior end 4 amounts to circa 2.5 mm, at the exterior end 3 it is also circa 2.5 mm and in between towards the centre of the object it increases to up to circa 4.5 mm.

The measured height of the intervertebral prosthesis 1 measured in FIG. 3 corresponds to the anatomical intervertebral disk space. In the centre of the convex intervertebral disk prosthesis 1, the height on both sides protrudes by circa 0.5 to 3.0 in comparison to the sections located on the rim.

In a spiral-wound, unloaded state of object 2—as shown in FIGS. 1 and 2 —there is a gap of 0.65 mm between each individual spiral turn.

Essentially object 2 consists of an envelope of polycarbonate urethane and/or silicon polycarbonate urethane as well as of a filling of polyvinyl alcohol hydrogel. Further suitable materials both for the envelope as well as for its filling may be obtained from the pending International Patent Application PCT/CHO1/00700.

FIG. 5 shows how injection point 9 is located in the area of the second end, which means nearly in the centre of the spiral-shaped object 2 and how it is recessed when compared with the upper spiral level 6.

FIG. 6 shows a possible variant of the exterior end 3 of object 2 with aids in the form of two grooves 11 located crossways to the central axis 5, which allow gripping the intervertebral disk prosthesis using a suitable insertion instrument, for example tongs.

FIG. 7 shows a second variant of the exterior end 3 of object 2 with aids, in this case in form of two shallow indentations 12 running crossways towards the central axis 5 as well as a slit 13 with a cylindrical counterdraft 14 positioned parallel to the central axis.

1. An intervertebral disk prosthesis or nuclear prosthesis (1) consisting of a longitudinal, flexibility-yielding object that is adapted to be wound in a spiral shape (2), with a first exterior end (3), a second interior end (4) and a longitudinal central axis (5), wherein
a cross-section (10) of the object (2) which is orthogonal towards the central axis (5) is reduced in size towards the second interior end (4).

2. The intervertebral disk prosthesis (1) according to claim 1, wherein the orthogonal cross-section (10) is continuously reduced in size.

3. The intervertebral disk prosthesis (1) according to claim 1, wherein the cross-section (10) of the object (2), which is orthogonal towards the central axis (5), is continuously reduced in size towards the first exterior end (3), preferably with the reduction occurring continuously.

4. The intervertebral disk prosthesis (1) according to claim 1, wherein a measured width of the object (2), running vertically towards the central axis (5), is reduced in size, when viewed from its center toward the exterior end (3), and wherein the measured width reduction occurs continuously.

5. The intervertebral disk prosthesis (1) according to claim 1, wherein a measured width of the object (2), running vertically towards the central axis (5), is reduced in size when viewed from its center toward the inner end (4), and wherein the measured width reduction occurs continuously.

6. The intervertebral disk prosthesis (1) according to claim 1, wherein a width of the object (2) in its center is wider by 50% to 500%, preferably wider by 100% to 300%, than at its interior and exterior ends (4, 3).

7. The intervertebral disk prosthesis (1) according to claim 1, wherein the object (2) in its spiral-wound state towards the central axis (5) features an upper spiral level (6) and a lower spiral level (7), which are both curved in convex shapes and are suitable for application to cover plates of two adjacent intervertebral disk spaces.

8. The intervertebral disk prosthesis (1) according to claim 1, wherein the object (2) in a spiral-wound unloaded state features a gap between the spirals.

9. The intervertebral disk prosthesis (1) according to claim 8, wherein the gap has a minimum width of 0.4 mm.

10. The intervertebral disk prosthesis (1) according to claim 8, wherien the gap has a maximum width of 1 mm.

11. The intervertebral disk prosthesis (1) according to claim 1, wherein the object (2) in a spiral-wound state, when viewed at the spiral level, features an oval or kidney-shaped shape, and having a surface between 250 to 750 mm² measured at the spiral level.

12. The intervertebral disk prosthesis (1) according to claim 1, wherein the object (2) contains a hydrogel.

13. The intervertebral disk prosthesis (1) according to claim 1, wherein the object (2) is manufactured using an injection-molding process and its injection point (9) is positioned in an area of the second end (4).

14. The intervertebral disk prosthesis (1) according to claim 13, wherein the injection point (9) is located in a recess vis-à-vis the upper spiral level (6).

15. The intervertebral disk prosthesis (1) according to claim 1, wherein the first end (3) is designed asymmetrically towards an interior of the spiral.

16. The intervertebral disk prosthesis (1) according to claim 1, wherein the object (2) is X-ray-opaque.

17. The intervertebral disk prosthesis (1) according to claim 1, wherein the object (2) contains X-ray-opaque components.

18. The intervertebral disk prosthesis (1) according to claim 1, wherein a last exterior spiral turn with a circumference of at least 3600 of the spiral-wound object (2) has a thinner cross-section than remaining spiral turns.

19. The intervertebral disk prosthesis (1) according to claim 1 wherein the exterior end (3) of the object (2) is fitted with aids (11, 12, 13, 14), which are suited for gripping the intervertebral disk prosthesis (1) using an insertion instrument.
